SEQUENCE LISTING (34 sequences)

SEQ ID NO: 1 - DNA, 25 bp, Artificial Sequence
  p21 Forward Primer
ttctgctgtctctcctcagatttct

SEQ ID NO: 2 - DNA, 21 bp, Artificial Sequence
  p21 Reverse Primer
ggattagggcttcctcttgga

SEQ ID NO: 3 - DNA, 22 bp, Artificial Sequence
  TaqMan Probe
ccactccaaacgccggctgatc

SEQ ID NO: 4 - DNA, 22 bp, Artificial Sequence
  GADD153 Forward Primer
agaaccaggaaacggaaacaga

SEQ ID NO: 5 - DNA, 20 bp, Artificial Sequence
  GADD153 Reverse Primer
tctccttcatgcgctgcttt

SEQ ID NO: 6 - DNA, 25 bp, Artificial Sequence
  SUMO-1 Forward Primer
gggtcagagaattgctgataatcat

SEQ ID NO: 7 - DNA, 22 bp, Artificial Sequence
  SUMO-1 Reverse Primer
ccccgtttgttcctgataaact

SEQ ID NO: 8 - DNA, 18 bp, Artificial Sequence
  Apaf-1 Forward Primer
tgcgctgctctgccttct

SEQ ID NO: 9 - DNA, 21 bp, Artificial Sequence
  Apaf-1 Reverse Primer
ccatgggtagcagctccttct

SEQ ID NO: 10 - DNA, 21 bp, Artificial Sequence
  Bcl-2 Forward Primer
catgtgtgtggagagcgtcaa

SEQ ID NO: 11 - DNA, 21 bp, Artificial Sequence
  Bcl-2 Reverse Primer
gccggttcaggtactcagtca

SEQ ID NO: 12 - DNA, 20 bp, Artificial Sequence
  PUMA Forward Primer
gggcccagactgtgaatcct

SEQ ID NO: 13 - DNA, 24 bp, Artificial Sequence
  PUMA Reverse Primer
acgtgctctctctaaacctatgca

SEQ ID NO: 14 - DNA, 19 bp, Artificial Sequence
  TaqMan Probe
ccccgccccatcaatccca

SEQ ID NO: 15 - DNA, 20 bp, Artificial Sequence
  NOXA Forward Primer
ctcaggaggtgcacgtttca

SEQ ID NO: 16 - DNA, 18 bp, Artificial Sequence
  NOXA Reverse Primer
ttccaagggcacccatga

SEQ ID NO: 17 - DNA, 19 bp, Artificial Sequence
  HRK Forward Primer
gggagcccagagcttgaaa

SEQ ID NO: 18 - DNA, 23 bp, Artificial Sequence
  HRK Reverse Primer
gcgctgtctttactctccacttc

SEQ ID NO: 19 - DNA, 23 bp, Artificial Sequence
  BIM Forward Primer
tccaggacagttggatattgtca

SEQ ID NO: 20 - DNA, 23 bp, Artificial Sequence
  BIM Reverse Primer
taaggagcaggcacagagaaaga

SEQ ID NO: 21 - DNA, 22 bp, Artificial Sequence
  BIK Forward Primer
tcctatggctctgcaattgtca

SEQ ID NO: 22 - DNA, 21 bp, Artificial Sequence
  BIK Reverse Primer
ggcaggagtgaatggctcttc

SEQ ID NO: 23 - DNA, 28 bp, Artificial Sequence
  BID Forward Primer
catacactttttctctttccatgacatc

SEQ ID NO: 24 - DNA, 20 bp, Artificial Sequence
  BID Reverse Primer
gggcatcgcagtagcttctg

SEQ ID NO: 25 - DNA, 22 bp, Artificial Sequence
  BAD Forward Primer
caggcctatgcaaaaagaggat

SEQ ID NO: 26 - DNA, 18 bp, Artificial Sequence
  BAD Reverse Primer
cgcaccggaagggaatct

SEQ ID NO: 27 - DNA, 18 bp, Artificial Sequence
  Bcl-Xs Forward Primer
ggcaggcgacgagtttga

SEQ ID NO: 28 - DNA, 27 bp, Artificial Sequence
  Bcl-Xs Reverse Primer
gttcccatagagttccacaaaagtatc

SEQ ID NO: 29 - DNA, 22 bp, Artificial Sequence
  BOK Forward Primer
tcacatgctggttgcttaatcc

SEQ ID NO: 30 - DNA, 19 bp, Artificial Sequence
  BOK Reverse Primer
gcacaaggaccccatcaca

SEQ ID NO: 31 - DNA, 21 bp, Artificial Sequence
  BAK Forward Primer
cacggcagagaatgcctatga

SEQ ID NO: 32 - DNA, 20 bp, Artificial Sequence
  BAK Reverse Primer
cccaattgatgccactctca

SEQ ID NO: 33 - DNA, 22 bp, Artificial Sequence
  BAX Forward Primer
tttctgacggcaacttcaactg

SEQ ID NO: 34 - DNA, 18 bp, Artificial Sequence
  BAX Reverse Primer
ggtgcacagggccttgag

(19) United States
(12) Patent Application Publication
(10) Pub. No.: US 2009/0215064 A1
(43) Pub. Date: Aug. 27, 2009

(54) QUANTITATIVE ASSESSMENT OF INDIVIDUAL CANCER SUSCEPTIBILITY BY MEASURING DNA DAMAGE-INDUCED MRNA IN WHOLE BLOOD

(75) Inventors: Masato Mitsuhashi, Irvine, CA (US); Hoda Anton-Culver, Laguna Beach, CA (US); Argyrios Ziogas, Irvine, CA (US); David Peel, Wroxall (GB)

Correspondence Address: KNOBBE MARTENS OLSON & BEAR LLP, 2040 MAIN STREET, FOURTEENTH FLOOR, IRVINE, CA, 92614, US

(73) Assignees: HITACHI CHEMICAL CO., LTD., Tokyo (JP); HITACHI CHEMICAL RESEARCH CENTER, INC., Irvine, CA (US); THE REGENTS OF THE UNIVERSITY OF CALIFORNIA, Oakland, CA (US)

(21) Appl. No.: 12/395,315

(22) Filed: Feb. 27, 2009

Related U.S. Application Data
(60) Provisional application No. 61/032,020, filed on Feb. 27, 2008.

(51) Int. Cl. C12Q 1/68

(52) U.S. Cl. 435/6

(57) ABSTRACT

Heparinized human whole blood from patients with invasive breast cancer, with (multiple primary) and without (single primary) a second primary cancer, and from unaffected controls was stimulated with 0.1-10 Gy of radiation and incubated at 37° C. for 2 hours. P21 and PUMA mRNA were then quantified. The results suggest that cancer susceptibility represented by the multiple primary cases was significantly related to over-reaction of p21 mRNA, and not PUMA.

### STATEMENT REGARDING FEDERALLY SPONSORED R&D

This study was funded in part by a grant from the Hereditary Breast Cancer Research Project (NIH # CA-58860-12). The government may have certain rights in the invention.

### PARTIES OF JOINT RESEARCH AGREEMENT

This research was carried out jointly by researchers from Hitachi Chemical Research Center, Inc., Irvine, Calif. 92617, USA and Epidemiology Division, Department of Medicine, University of California-Irvine, Irvine, Calif. 92697, USA.

### REFERENCE TO SEQUENCE LISTING TABLE, OR COMPUTER PROGRAM LISTING

A Sequence Listing is provided herewith.

### BACKGROUND

1. Field of the Invention

The present disclosure relates to a method for determining cancer susceptibility by quantifying DNA damage-induced mRNA in whole blood.

2. Description of the Related Art

Cancer is caused by DNA mutation from exposure to DNA-damaging agents such as ionizing radiation, ultraviolet light, carcinogens, and free radicals, and by certain viral infections. Although cells successfully repair the majority of DNA damage, accumulation of uncured or miscured DNA damage at critical places within the genome may lead to the development of cancer. Thus, cancer susceptibility depends on the balance between DNA damage and corresponding cellular responses in a given individual. In fact, poor DNA damage response in ataxia telangiectasia (see Paterson, M. C. et al., Nature, 260, 444-47 (1976)) is known to frequently lead to the development of cancer. We first identified appropriate mRNA markers for DNA damage response and then applied the results to a clinical feasibility study.

### SUMMARY

Heparinized human whole blood from patients with invasive breast cancer, with (multiple primary) and without (single primary) a second primary cancer, and from unaffected controls was stimulated with 0.1-10 Gy of radiation and incubated at 37° C. for 2 hours. P21 and PUMA mRNA were then quantified. The results suggest that cancer susceptibility represented by the multiple primary cases was significantly related to over-reaction of p21 mRNA, and not PUMA.

### BRIEF DESCRIPTION OF THE DRAWINGS

FIG. 1 shows the relative increase of various radiation-induced mRNAs as compared to control samples. The FIG. 1 inset shows the relative amounts of p21 and PUMA mRNA induced over time with and without exposure to 10 Gy radiation.

FIG. 2 shows the relative increase in radiation-induced p21 and PUMA mRNA as compared to control samples for various levels of radiation exposure.

### DETAILED DESCRIPTION OF THE PREFERRED EMBODIMENTS

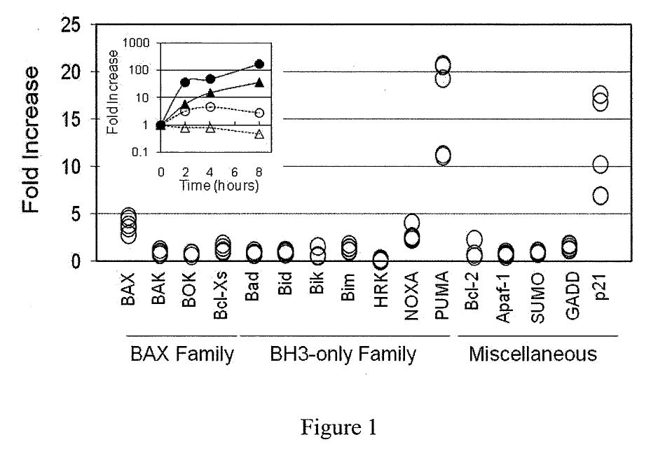

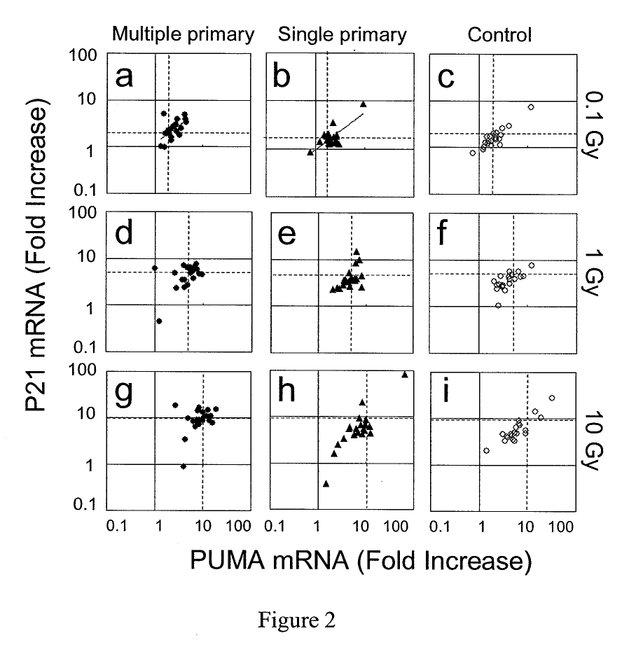

Blood samples were collected from healthy adult donors (approved by the institutional review board (IRB) of APEX Research Institute, Tustin, Calif.). After treating the samples with 30 Gy of radiation (cesium-137), we first screened for expression of various mRNAs using a method we recently developed (see Mitsuhashi, M., Endo, K. & Shinagawa, A., Clin. Chem., 52, 634-42 (2006); Mitsuhashi, M., Clin. Chem., 53, 148-49 (2007)) with SYBR green real time PCR (see Morrison, T. B., Weis, J. J. & Wittwer, C. T., Biotechniques, 24, 954-62 (1998)) (FIG. 1) and TaqMan real time PCR (see Holland, P. M. et al., Proc. Natl. Acad. Sci. U.S.A., 88, 7276-80 (1991)) (FIG. 1 inset, FIG. 2), using triplicate 50 μL aliquots of heparinized whole blood. Primers and TaqMan probes were designed by Primer Express (Applied Biosystem, Foster City, Calif.) and HYBsimulator (RNAture, Irvine, Calif.) (see Mitsuhashi, M. et al, Nature, 367, 759-61 (1994)) (Table 3). Oligonucleotides were synthesized by IDT (Coralville, Iowa). The specific primer and probe sequences used in these studies are shown in Table 1:

TABLE 1
Primer and probe sequences used.

Sequences (5′-3′)

| | | | | |
|---|---|---|---|---|
| | | SEQ | | SEQ |
| | | ID | | ID |
| mRNA | Forward primer | NO. | Reverse primer | NO. |

| | | | | |
|---|---|---|---|---|
| p21 | TTCTGCTGTC TCTCCTCAGA TTTCT | 1 | GGATTAGGGC TTCCTCTTGG A | 2 |

TaqMan probe: FAM-CCACTCCAAA CGCCGGCTGA TC-TAMRA (SEQ ID NO. 3)

| | | | | |
|---|---|---|---|---|
| GADD153 | AGAACCAGGA AACGGAAACA GA | 4 | TCTCCTTCAT GCGCTGCTTT | 5 |
| SUMO-1 | GGGTCAGAGA ATTGCTGATA ATCAT | 6 | CCCCGTTTGT TCCTGATAAA CT | 7 |
| Apaf-1 | TGCGCTGCTC TGCCTTCT | 8 | CCATGGGTAG CAGCTCCTTC T | 9 |
| Bcl-2 | CATGTGTGTG GAGAGCGTCA A | 10 | GCCGGTTCAG GTACTCAGTC A | 11 |
| PUMA | GGGCCCAGAC TGTGAATCCT | 12 | ACGTGCTCTC TCTAAACCTA TGCA | 13 |

TaqMan probe: FAM-CCCCGCCCCA TCAATCCCA-TAMRA (SEQ ID NO. 14)

| | | | | |
|---|---|---|---|---|
| NOXA | CTCAGGAGGT GCACGTTTCA | 15 | TTCCAAGGGC ACCCATGA | 16 |
| HRK | GGGAGCCCAG AGCTTGAAA | 17 | GCGCTGTCTT TACTCTCCAC TTC | 18 |
| BIM | TCCAGGACAG TTGGATATTG TCA | 19 | TAAGGAGCAG GCACAGAGAA AGA | 20 |
| BIK | TCCTATGGCT CTGCAATTGT CA | 21 | GGCAGGAGTG AATGGCTCTT C | 22 |
| BID | CATACACTTT TTCTCTTTCC ATGACATC | 23 | GGGCATCGCA GTAGCTTCTG | 24 |
| BAD | CAGGCCTATG CAAAAAGAGG AT | 25 | CGCACCGGAA GGGAATCT | 26 |
| Bcl-Xs | GGCAGGCGAC GAGTTTGA | 27 | GTTCCCATAG AGTTCCACAA AAGTATC | 28 |
| BOK | TCACATGCTG GTTGCTTAAT CC | 29 | GCACAAGGAC CCCATCACA | 30 |
| BAK | CACGGCAGAG AATGCCTATG A | 31 | CCCAATTGAT GCCACTCTCA | 32 |
| BAX | TTTCTGACGG CAACTTCAAC TG | 33 | GGTGCACAGG GCCTTGAG | 34 |

In these studies, heparinized human whole blood samples from five healthy individuals were stimulated with or without 30 Gy of radiation (cesium-137) and incubated at 37° C. for 4 hours. After incubation, triplicate 50 μL aliquots of whole blood were used to quantify various mRNAs by a method we recently developed (see Mitsuhashi, M., Endo, K. & Shinagawa, A., Clin. Chem., 52, 634-42 (2006); Mitsuhashi, M., Clin. Chem., 53, 148-49 (2007)) with SYBR green real time PCR (see Morrison, T. B., Weis, J. J. & Wittwer, C. T., Biotechniques, 24, 954-62 (1998)). Each gene was amplified individually. The cycle threshold (Ct)—the cycle of PCR that generates certain amounts of PCR products (fluorescence)—was determined using analytical software (SDS, Applied Biosystems). The melting curve was analyzed in each case to confirm that the PCR signals were derived from a single PCR product. The Ct values of drug-treated triplicate samples were subtracted from the mean Ct values of control samples to calculate ΔCt, and the fold increase was calculated as 2^{-ΔCt}.

The results are shown in FIG. 1, expressed as the fold increase of induced mRNA, using the values for unexposed samples as the controls. We found that p21 (cyclin dependent kinase inhibitor 1A) (see Han, J. et al., Proc. Natl. Acad. Sci. U.S.A., 98, 11318-23 (2001)) and PUMA (Bcl-2 binding component 3 (bbc3)) (see Yu, J. et al., Mol. Cell, 7, 673-82 (2001); Nakano, K. & Vousden, K. H., Mol. Cell, 7, 683-94 (2001); Villunger, A. et al., Science, 302, 1036-38 (2003)) were the most prominent and universal marker mRNAs in blood leukocytes (FIG. 1). As can be seen in FIG. 1, BAX and NOXA were also induced; however the increase was smaller than that of p21 and PUMA (FIG. 1).

Kinetic studies on p21 and PUMA mRNA were conducted using TaqMan real time PCR (see Holland, P. M. et al., Proc. Natl. Acad. Sci. U.S.A., 88, 7276-80 (1991)) with the values at time=0 as controls. The results are shown in the inset of FIG. 1. Each symbol represents the mean of p21 (: 10 Gy, ◯: control) and PUMA (▴: 10 Gy, Δ: control) mRNA from triplicate aliquots of whole blood derived from a single individual. After radiation exposure (10 Gy), p21 levels were significantly increased as compared to the controls (FIG. 1 inset, ). Unlike p21, base line PUMA expression was unchanged during 8 hours of incubation at 37° C. (FIG. 1 inset, Δ). However, it was significantly increased after radiation exposure (FIG. 1 inset, ▴). The levels of p21 and PUMA mRNA increased rapidly, with the peak at around 2-4 hours (FIG. 1 inset).

We hypothesized that cancer susceptibility might be linked to hypo-functions of p21 and/or PUMA, based on our knowledge of ataxia telangiectasia (see Paterson, M. C. et al, Nature, 260, 444-47 (1976)). To test this hypothesis, we undertook studies to evaluate the blood from control and cancer patients for inducibility of p21 and PUMA mRNA after radiation exposure. After obtaining approval for the study protocol from the IRB of the University of California-Irvine (UCI), we identified 38 cases in the local cancer registry where the patient had both invasive breast cancer and a second primary cancer (multiple primary cases (MP)). After initial contact, we recruited 21 women to participate in the study. We then selected a second cancer group of single primary cases (SP) (n=21) and unaffected control cases (UC) (n=20) with similar age and ethnicity distributions. Table 2 provides demographic and tumor characteristics of the recruited participants, and Table 3 shows their white blood cell (WBC) counts:

TABLE 2
Demographic and tumor characteristics of recruited participants.

| MP (n = 21) | | SP (n = 21) | | UC (n = 20) | |
|---|---|---|---|---|---|
| N | % | N | % | N | % |

Race/Ethnicity

| | | | | | | |
|---|---|---|---|---|---|---|
| NH White | 20 | 95.2% | 18 | 85.7% | 20 | 100.0% |
| Hispanic | 0 | 0.0% | 3 | 14.3% | 0 | 0.0% |
| Asian | 1 | 4.8% | 0 | 0.0% | 0 | 0.0% |

Age Now

| | | | | | | |
|---|---|---|---|---|---|---|
| 40-49.9 | 2 | 9.5% | 1 | 4.8% | 3 | 15.0% |
| 50-59.9 | 6 | 28.6% | 8 | 38.1% | 9 | 45.0% |
| 60-69.9 | 10 | 47.6% | 10 | 47.6% | 3 | 15.0% |
| 70-79.9 | 3 | 14.3% | 2 | 9.5% | 5 | 25.0% |

Age @ Invasive Breast Cancer

| | | | | |
|---|---|---|---|---|
| 40-49.9 | 3 | 14.3% | 4 | 19.0% |
| 50-59.9 | 7 | 33.3% | 13 | 61.9% |
| 60-69.9 | 8 | 38.1% | 4 | 19.0% |
| 70-79.9 | 3 | 14.3% | 0 | 0.0% |

Stage of Invasive Breast Cancer

| | | | | |
|---|---|---|---|---|
| Localized | 13 | 61.9% | 14 | 66.7% |
| Regional, Lymph Nodes | 6 | 28.6% | 7 | 33.3% |
| Unknown | 2 | 9.5% | 0 | 0.0% |

Histology of Invasive Breast Cancer

| | | | | |
|---|---|---|---|---|
| Papillary Cancer | 1 | 4.8% | 0 | 0.0% |
| Infiltrating Duct Cancer | 12 | 57.1% | 17 | 81.0% |
| Lobular Cancer | 2 | 9.5% | 2 | 9.5% |
| Ductal & Lobular Cancer | 3 | 14.3% | 2 | 9.5% |
| Infilt. Duct w/Other Cancer | 3 | 14.3% | 0 | 0.0% |

No. of Years between Invasive Breast & Other Cancer

| | | |
|---|---|---|
| 0-4.9 | 7 | 33.3% |
| 5-9.9 | 5 | 23.8% |
| 10-14.9 | 8 | 38.1% |
| 15-19.9 | 1 | 4.8% |

Type of Other Cancer (5 have >1 other cancer)

| | | |
|---|---|---|
| Ovary | 2 | 7.7% |
| Lung | 2 | 7.7% |
| Breast | 4 | 15.4% |
| Endometrium | 5 | 19.2% |
| Thyroid | 1 | 3.8% |
| Rectum | 4 | 15.4% |
| Melanoma | 5 | 19.2% |
| Lymph Nodes | 2 | 7.7% |
| Kidney | 1 | 3.8% |

TABLE 3
White blood cell (WBC) counts of recruited participants.

| | WBC count (StDev) |
|---|---|
| Control | 6.3 (1.29) |
| Single Primary | 5.58 (1.71) |
| Multiple Primary | 5.23 (1.29) |

We dispatched clinical nurses to the participants' homes to complete questionnaires, and blood was drawn in two tubes from each participant, one for a complete blood count (see Table 2) and the other for mRNA analysis. Blood samples were immediately transferred to the laboratory at 4° C. The blood was treated the same day with radiation (2 hours at 37° C.), and the samples were then frozen at −80° C.

Specifically, the heparinized human whole blood samples from invasive breast cancer with or without a second primary cancer, or unaffected control (◯, n=20, FIGS. 2c, 2f, 2i) were stimulated with 0.1 (FIGS. 2a, 2b, 2c), 1 (FIGS. 2d, 2e, 2f), and 10 Gy (FIGS. 2g, 2h, 2i) of radiation in quadruplicate, and incubated at 37° C. for 2 hours. P21 and PUMA mRNAs were then quantified with TaqMan real time PCR (see Holland, P. M. et al., Proc. Natl. Acad. Sci. U.S.A., 88, 7276-80 (1991)). The fold increase was calculated using the values of unexposed samples.

The results are reported in FIG. 2. In a, d, and g, the results obtained using blood from each of the 21 individual multiple primary (MP) cancer patients are shown using the o symbol. In b, e, and h, the results obtained using blood from each of the 21 single primary (SP) cancer patients are shown using the ▴ symbol. Finally, in c, f, and i, the results obtained using blood from each of the 20 unaffected control (UC) individuals are shown using the ◯ symbol. In a, b and c, brood was stimulated with 0.1 Gy; in d, e, and f blood was stimulated with 1 Gy; and in g, h, and i, blood was stimulated with 10 Gy of radiation. The dashed lines are presented solely as reference points, and represent fold increases of two (a, b, c), five (d, e, f), and ten (g, h, i) for both p21 and PUMA.

As shown in FIG. 2, both p21 and PUMA mRNA increased in a dose dependent manner after radiation exposure. However, large variations within each group were observed, and a t-test did not reveal any significant differences among the MP, SP, and UC cases for radiation-induced increase in PUMA mRNA. On the other hand, the median increase for 10 Gy-induced p21 (10.1±4.0 fold increase) was significantly higher in MP cases (p=0.04) than in UC cases (6.8±5.7).

The population density was shifted upward for radiation-induced p21 mRNA in MP cases for all doses of radiation as compared to the other two groups. For example, three (14%) and four (20%) individuals in SP and UC cases respectively showed a greater than two-fold p21 mRNA induction at 0.1 Gy, whereas the percentage was significantly higher in MP cases (57%)—this difference was statistically significant (p=0.004 (MP v. SP), p=0.01 (MP v. UC) by χ² test, respectively) (FIG. 1, a-c). Similarly, at 1 Gy (FIG. 1, d-f), four and three individuals in SP and UC cases respectively showed a greater than five-fold increase in p21 mRNA, whereas ten individuals showed a similar increase in MP cases (p=0.05 (MP v. SP), p=0.02 (MP v. UC) by χ² test, respectively). At 10 Gy (FIG. 1, g-i), only two and three individuals in SP and UC cases respectively showed a greater than ten-fold increase in p21 mRNA, whereas nine individuals showed a similar increase in MP cases (p=0.01 (MP v. SP), p=0.05 (MP v. UC) by χ² test, respectively). Thus, there was an increased number of patients in the MP group showing an increase in inducibility of p21 mRNA by all three doses of radiation. There was no significant difference between SP and UC cases (p>0.3).

As discussed above, we had initially hypothesized that cancer susceptibility might be linked to hypo-functions of p21 and/or PUMA, based on our knowledge of ataxia telangiectasia (see Paterson, M. C. et al., Nature, 260, 444-47 (1976)). Surprisingly, the results suggested that cancer susceptibility is related to the over-reaction of p21 mRNA only, and not PUMA (see FIG. 2). While not wishing to be bound by any particular theory for this result, it is believed since p21 is responsive to cell cycle arrest, which serves as a foundation for various DNA repair mechanisms, over-reaction of p21 may increase the chance of the miscure of DNA. By contrast, over-function of PUMA is less likely to be linked to cancer susceptibility, because increased PUMA function causes cells to die by apoptosis without carrying mutated DNA to daughter cells. Both p21 and PUMA mRNA are controlled by the transcription factor p53 (see Paterson, M. C. et al., Nature, 260, 444-47 (1976); Iyer, N. G. et al., Proc. Natl. Acad. Sci. U.S.A., 101, 7386-91 (2004)), and these two mRNAs were in fact correlated with each other (the r² value for all data combined was 0.821). The discrepancies in radiation-induced mRNA among the three groups may indicate an additional consideration important to understanding the p53-p21-PUMA axis.

Cancer susceptibility is currently analyzed extensively from a genomics perspective in order to identify specific single nucleotide polymorphisms (SNPs) (see Karlan, B. Y., Berchuck, A. & Mutch, D., Obstet. Gynecol., 110, 155-67 (2007); Oldenburg, R. A. et al., Crit. Rev. Oncol. Hematol., 63, 125-49 (2007); Naccarati, A. et al., Mutat. Res., 635, 118-45 (2007)). However, we still do not know whether yet-to-be discovered second or third SNPs will compensate for or aggregate the effects of a given SNP. By contrast, we quantified the levels of normally existing mRNA without considering SNPs in p21 and PUMA. The hyper-function of p21 mRNA that we found may result from an SNP in p53 or other related genes. Alternatively, it may be related to the strength of each participant's antioxidant levels, which protects against DNA damage. We have thus generated a unique model for cancer susceptibility research as a screening tool for various downstream molecular assays.

All references cited herein are expressly incorporated by reference.

What is claimed is:

1. A method of determining cancer susceptibility in an individual, comprising
(a) obtaining a sample comprising leukocytes from the individual;
(b) determining the amount of p21 mRNA in the sample;
(c) exposing said sample to a source of DNA damage;
(d) determining the amount of p21 mRNA in the sample after said exposure;
(e) determining the amount of increase in p21 mRNA induced by the DNA damage by subtracting the amount obtained in step (b) from the amount obtained in step (d);
(f) comparing the determined amount of increase in p21 mRNA with an amount of increase in p21 mRNA expected to be induced in a sample from a control patient who is not susceptible to cancer, wherein an increase in induced p21 mRNA that is greater than the increase in induced p21 mRNA in a sample from a patient who is not susceptible to cancer is indicative of a greater susceptibility to cancer.

2. The method of claim 1, wherein the amount expected to be induced in a sample from a control patient is determined as an average of the increases in p21 mRNA obtained from samples from a plurality of patients who have never had cancer.

3. The method of claim 1, wherein said mRNA is induced in whole blood.

4. The method of claim 3, wherein said whole blood is heparinized human whole blood.

5. The method of claim 1, wherein said source of DNA damage is radiation.

6. The method of claim 5, wherein the radiation source is cesium-137.

7. The method of claim 5, wherein the radiation source exposes the sample to about 0.1, 1, or 10 Gy of radiation.

8. The method of claim 2, wherein greater than a 20% increase in p21 mRNA compared to said expected increase is indicative of a greater susceptibility to cancer.

9. The method of claim 2, wherein greater than a 50% increase in p21 mRNA compared to said expected increase is indicative of a greater susceptibility to cancer.

10. The method of claim 2, wherein greater than a 75% increase in p21 mRNA compared to said expected increase is indicative of a greater susceptibility to cancer.

11. The method of claim 2, wherein greater than a 100% increase in p21 mRNA compared to said expected increase is indicative of a greater susceptibility to cancer.

12. The method of claim 2, wherein greater than a 200% increase in p21 mRNA compared to said expected increase is indicative of a greater susceptibility to cancer.
